(19) United States
(12) Patent Application Publication
(10) Pub. No.: US 2008/0140080 A1
(43) Pub. Date: Jun. 12, 2008

(54) Instrument to make openings in bone in the form of a bone lid

(76) Inventor: Bernhard Strehl, Neu-Seiesberg (AT)

Correspondence Address: DR. BERNHARD STREHL, SANDGRUBENWEG 6, NEU-SEIERSBERG, A - 8055, omitted

(21) Appl. No.: 11/609,454

(22) Filed: Dec. 12, 2006

(51) Int. Cl. A61B 17/58

(52) U.S. Cl. 606/83

(57) ABSTRACT

The invention relates to an instrument for making openings in bone in form of a bone lid. The object of invention is that several cutting pins are carried along a hypotrochoid and in this way the instrument by utilizing a rotating drive mechanism produces the opening and the bone lid in a shape resembling a equilateral polygon with rounded edges.

### BACKGROUND OF THE INVENTION

The invention relates to an instrument for making openings in bone in form of a bone lid, which generates the opening and the bone lid in a shape resembling a equilateral polygon with rounded edges. Such a bone saw or bone mill can be employed advantageously in all situations, where an opening shall be made in a bony hollow space by removing one compact piece of bone (the so called bone lid) and this opening shall be closed again with the removed piece of bone. The instrument is driven with the aid of a rotating drive mechanism.

For producing openings in bone and bone lids a variety of different means are known (for instance DE4029676A1, EP0962192A1, GB110179A, GB941420A, GB1455566A, GB2420979A, U.S. Pat. No. 4,768,504 A1, U.S. Pat. No. 5,201,749 A1, WO9710765A1). These can basically be categorized in the following four groups:

A) Small circular saws guided by hand with either rotating or angularly oscillating saw blades. With the aid of these circular saws only straight cuts into the bone can be produced. In order to make an opening into the bone or a bone lid respectively, several straight cuts must be performed. Usually these cuts are arranged in the shape of a rectangle.

B) Small oscillating jigsaws, which are also guided by hand. By means of a saw blade oscillating up and down contours can be sawn into bone having a minimal radius. In order to penetrate the bone for the first time either a small hole is pre-drilled into which the jigsaw is inserted or at the beginning of the cut the jigsaw is placed sloping to the surface of the bone in a very shallow angle and penetrates the bone in a rasping manner. After the first penetration through the bone the jigsaw is positioned perpendicular and the desired contour can be cut out.

C) Cylindrical hole-saws having their teeth arranged along the front edge of the cylindrical part and rotating around a center shaft. With this type of hole-saws exact circular holes are achieved. Some embodiments of such hole-saws have a the centering shaft to be inserted into a pre-bored center boring.

D) Ultrasound-applicators oscillating with ultrasound, which abrade the bone in small quantities in a rubbing or grinding manner. The shape of the opening is either determined by the shape of the applicator or the ultrasound-applicator has the shape of a blade or a miller and is guided by hand along the desired contour. The surface of these ultrasound-applicators can be coated with an abrading medium or have a multitude of small sharp cutting edges.

To A) Employment of circular saws has the following disadvantages:
At the corners of the rectangle the cuts have to be longer then the actual sides of the rectangle in order to cut through the full thickness of the bone. Therefore at the corner points the straight cuts resemble crosses and more bone is cut than necessary for the opening.
The saw blade of the circular saw must be inserted into the bone deeper then the actual bone thickness in order to get a good cut. Therefore tissue structures just below the bone are at risk of being damaged.
Since the rotating axis of the circular saw is parallel to the surface of the bone and the driving mechanism needs space the circular saw is difficult to guide particular in confined situations. In some cases the bone lid can not be placed where it is desired.

To B) Employment of oscillating jigsaws has the following disadvantages:
The pre-bored hole made to insert the saw blade must be rather large in diameter entailing a large defect in the bone. Inserting the saw blade in the way of starting in a shallow angle can not be carried out on uneven surfaces of bone and needs a lot of space anyway.
The depth that is reached by the oscillating saw blade is difficult to be controlled and there exists a risk of damaging tissue structures just below the bone.
The radii of the contour to be cut out can not be smaller than a certain minimum value determined by the dimensions of the blade.

To C) Employment of cylindrical hole-saws has the following disadvantages:
This type of hole saw produces openings and lids with exact circular shape. Since some bone is lost in the form of small shavings the diameter of the lid is smaller than the diameter of the opening. Therefore a small sickle shaped residual opening remains when the lid is reinserted and the bone lid has contact to the edge of opening only in one point. This impedes heeling in of the bone lid.

To D) Employment of ultrasound-applicators has the following disadvantages:
Progress is rather slow and the cutting edges become clogged by small particles of bone and surrounding soft tissue.

Object of the invention is to make openings in bone and bone lids and to shape the contour of the opening in such a way that even after the inevitable loss of bone substance due to the cutting process the bone lid after re-insertion touches the edges of the opening not only in some points but along a continuous edge as long as possible. Additionally a fast way of working should be possible. The depth of penetration of the device should first be easily controlled and second as small as possible. Finally the device should be driven by a rotating drive mechanism as those used frequently in surgical and dental disciplines.

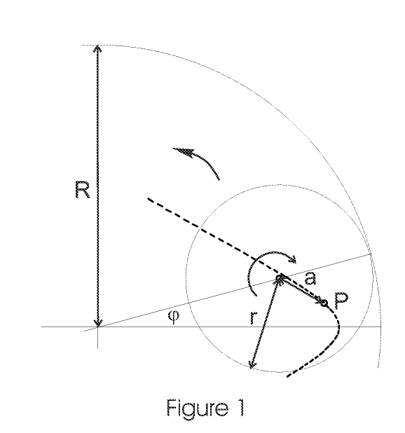

The foregoing objectives are achieved by the present invention in the following way:
1. Production of the cutting channel is achieved by several cutting pins guided along the same hypotrochoid around a central axis
2. Here the hypotrochoid is defined by the following equations and parameters (see also FIG. 1)

x((φ)=R[(1-1/V_{R}).cos((φ)+(Vₐ/V_{R}).cos((V_{R}-1). φ)]

y((φ)=R[(1-1/V_{R}).sin((φ)−(Vₐ/V_{R}).sin((V_{R}-1). (φ)]

φ: angle to the center the rolling circle (curve-parameter)

R: Radius of the fixed circle

V_{R}=R/r: Ratio between radius R of the fixed circle and radius r of the rolling circle

Vₐ=a/r: Ratio between the distance a of the curve-generating point P from the center of the rolling circle and the radius r of the rolling circle
3. For the present invention the parameter V_{R} is integer and has a value from 3 to 8. The parameter Vₐ has a value between 0,1 and 0,9.
4. The actual cutting line in the bone in the shape of such a hypotrochoid is achieved by an arrangement of several cogwheels with external toothing rolling inside a larger hollow cylinder with an internal toothing.
5. These smaller cogwheels are installed on a revolving support similar to a planetary gear. On this support they can rotate freely.
6. The larger cogwheel with internal toothing stands still. The smaller cogwheels are driven be the rotation of the revolving support.
7. The revolving support has a driving shaft driven by an external driving mechanism and on the opposite side a centering axis as axial guidance of the instrument.
8. On each of the smaller cogwheels one cutting pin is mounted. All cutting pins have the same shape and the same dimensions.
9. Certain embodiments of the cutting pins have at their front side a defined cutting surface and cutting edge. Other embodiments have their front side covered with an abrasive substance or have a multitude of small jagged teeth.
10. The distance between the center of the cutting edges of the pins and the center of the smaller cogwheel they are mounted on corresponds to “a” of the parameter Vₐ in point two of this description. The angular locations of the cutting pins on each of the smaller cogwheels in combination with the position of engagement of the smaller cogwheels into the larger cogwheel are chosen in such a way that all cutting pins run along the same hypotrochoid. Therefore all cutting pins produce the same cutting channel.
11. The number of teeth of the larger cogwheel with internal toothing is equal to or a multiple of the lowest common multiplier of V_{R}, the number of smaller cogwheels and the number of teeth of these smaller cogwheels. In this way the relative position of the cutting pin on each smaller cogwheel in relation to the toothing of these wheels is the same for all of the smaller cogwheels.

Further advantages, features and details of the invention will become clear from the following drawings and descriptions thereof, in which:

FIG. 1: shows a sketch of the relations for a hypotrochoid. A rolling circle with radius r rolls along the inside of a fixed circle with radius R without gliding. The point P generating the curve is part of the rolling circle and has distance “a” from the center of the rolling circle. For the invention a is always smaller than r.

As already done under point 2 of the description two ratios should be defined:

V_{R}=R/r: Ratio between radius R of the fixed circle and radius r of the rolling circle

Vₐ=a/r: Ratio between the distance “a” of the curve-generating point from the center of the rolling circle and the radius r of the rolling circle

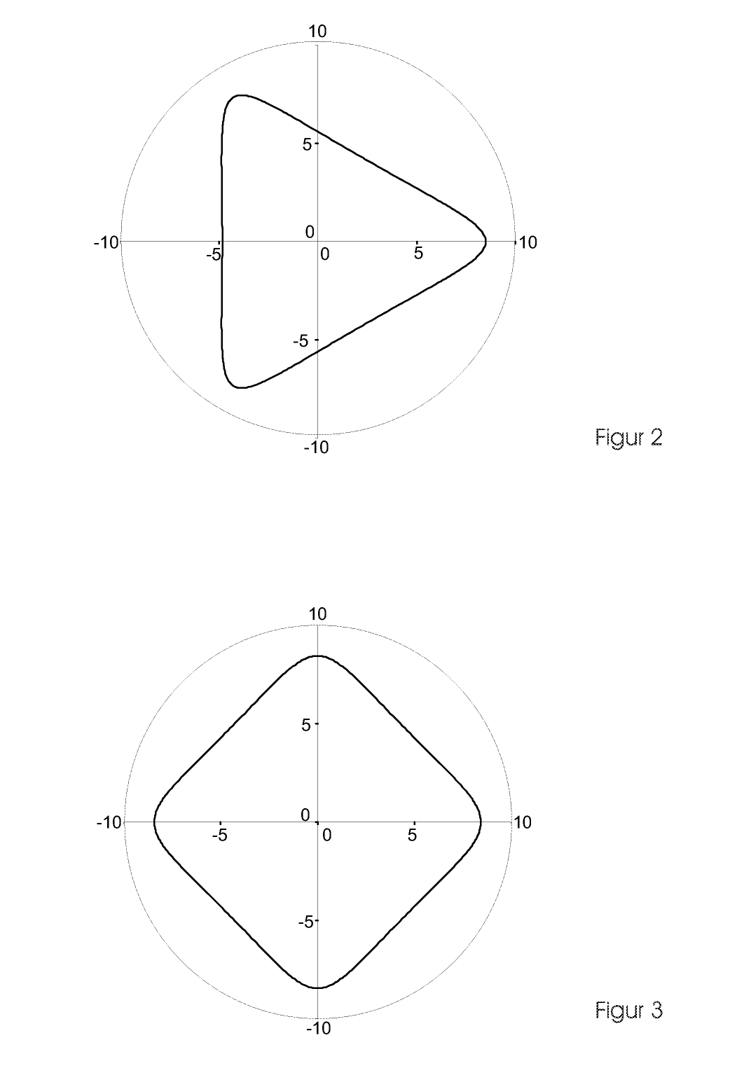

FIG. 2: shows an example of a specific hypotrochoid which is used in one particular preferred embodiment of the invention; R=10, V_{R}=3 und Vₐ=11/20

FIG. 3: shows another example for such a hypotrochoid; R=10, V_{R}=4 und Vₐ=15/40

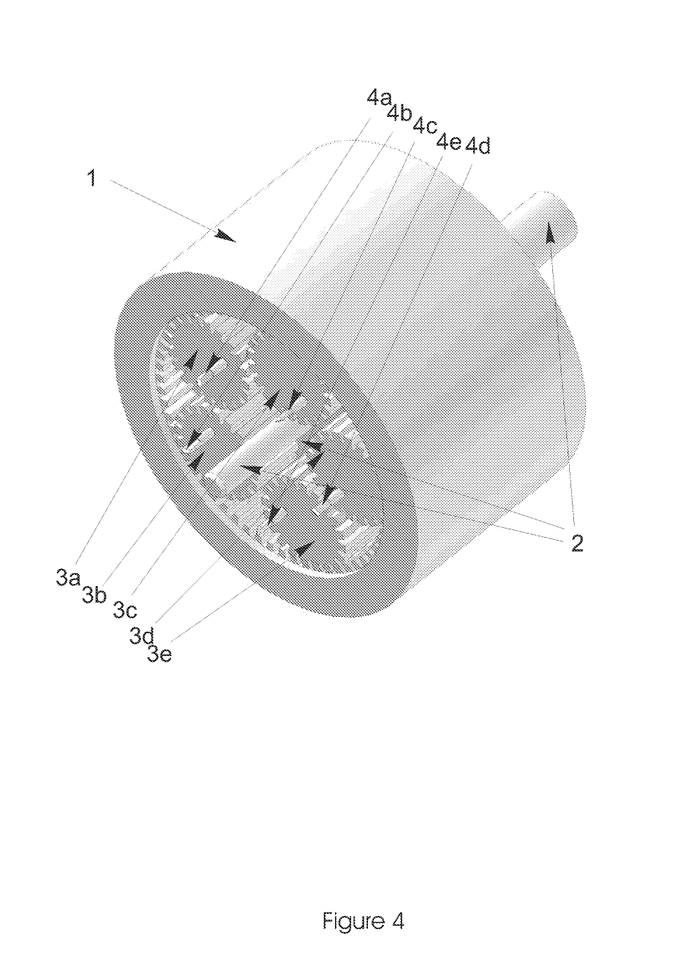

FIG. 4: shows a preferred embodiment of the device for making openings in bone in form of a bone lid. In this case V_{R} is 3 and the number of smaller cogwheels is 5.

A hollow cylinder open only on one end side (1) is provided with an internal toothing. This hollow cylinder is realization of the fixed circle for the generation of the hypotrochoid. The pitch radius of the internal toothing corresponds to R the radius of the fixed circle (see point 2 of the description). The hollow cylinder has a hole on the closed end side trough which the axis of the revolving support (2) sticks through. For specific embodiments, this axis is secured against axial movement by mechanical elements, for other embodiments the revolving support can slide axially in the hole. A second axis or pin on the opposite side of the revolving support centers the device in a pre-bored guiding hole in the bone.

Several (in this case five) smaller cogwheels (3a, 3b, 3c, 3d, 3e) are attached to the revolving support; they can rotate freely on the support. Their toothings engage with the internal toothing of the hollow cylinder (1). The pitch radius of these smaller cogwheels corresponds to “r” the radius of the rolling circle (see point 2 of the description).

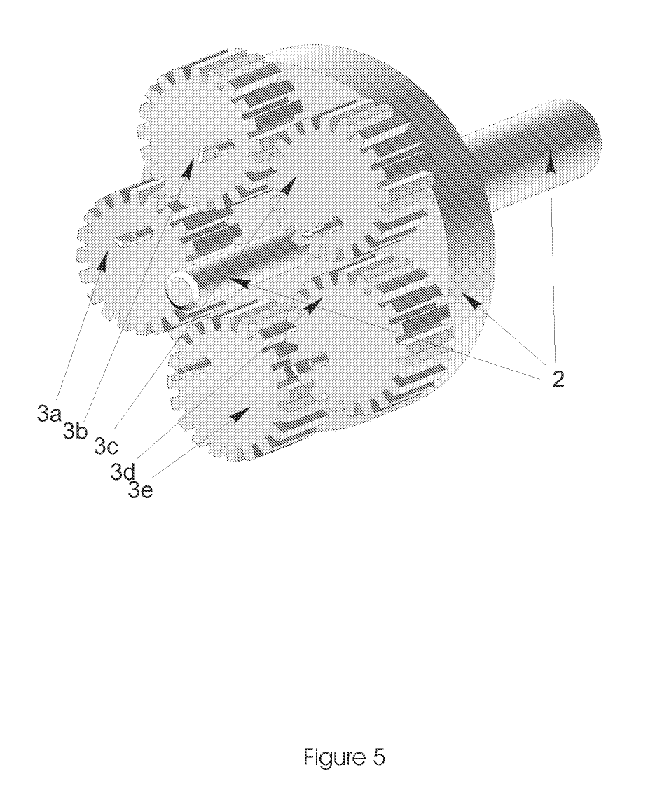

Each of the smaller cogwheels has one hole into which a cutting pin is pressed in; one for each smaller cogwheel (4a, 4b, 4c, 4d, 4e). Examples for different shapes of the cutting pin are shown in FIG. 5 and are described there.

When the axis of the revolving support (2) is driven by an external drive mechanism, the smaller cogwheels (3a, 3b, 3c, 3d, 3e) roll along the internal toothing of the hollow cylinder (1) and the cutting pins (4a, 4b, 4c, 4d, 4e) perform a movement along a hypotrochoid determined by the parameters V_{R} and V_{A}.

For all cutting pins and their corresponding smaller cogwheels the distance between the center of the cutting edge of the pin and the center of the smaller cogwheel is the same and corresponds to “a” of point 2 of the description and FIG. 1. The angular position of the hole in the cogwheel in relation to the toothing is the same for all pins or cogwheels respectively. All smaller cogwheels engage with the internal toothing of the hollow cylinder in such a way that the attached cutting pins run along the same hypotrochoid and therefore cut the same cutting channel. This is possible due to the number of teeth of the internal toothing of the hollow cylinder, as mentioned above.

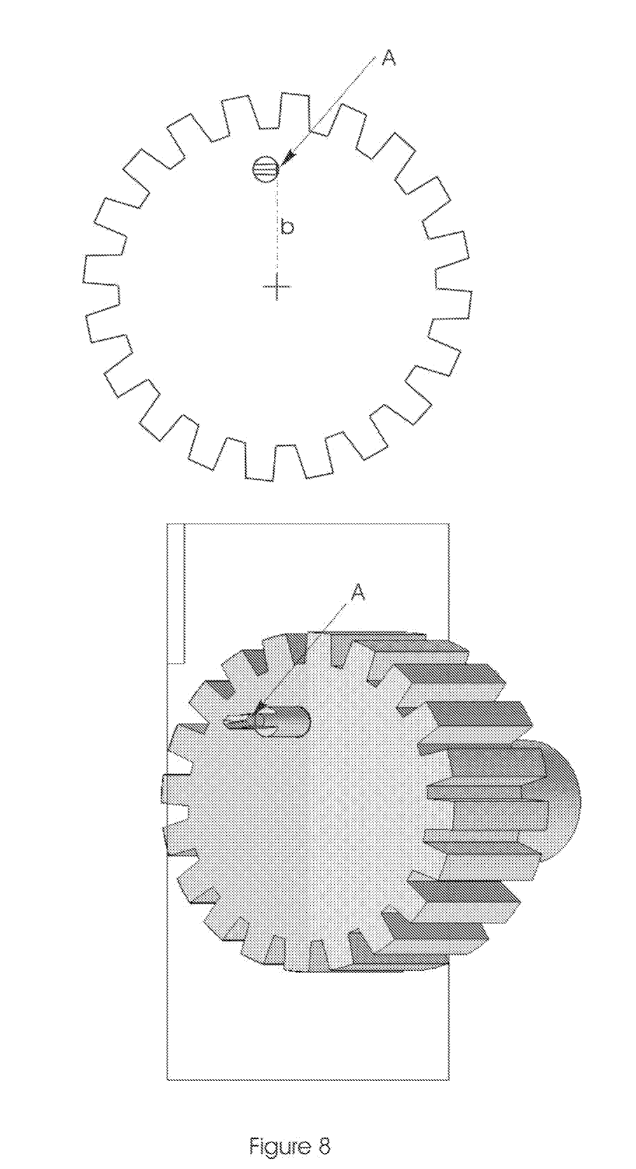

All cutting pins have the same shape and same dimensions. They are all pressed into the smaller cogwheels perpendicular to the end surfaces of the cogwheel, i.e. parallel to the axis of rotation of the smaller cogwheels. They are pressed into the smaller cogwheels in such a way that projections of the cutting surfaces are always collinear to the radial beams from the center of the smaller cogwheels. Therefore the cutting surfaces cut always perpendicular to the rotational movement of the smaller cogwheels. This fact is shown in detail in FIG. 8.

FIG. 5: shows the revolving support (2) together with the rolling cogwheels (3a, 3b, 3c, 3d, 3e) attached to it. These cogwheels can rotate freely on the support around their axis.

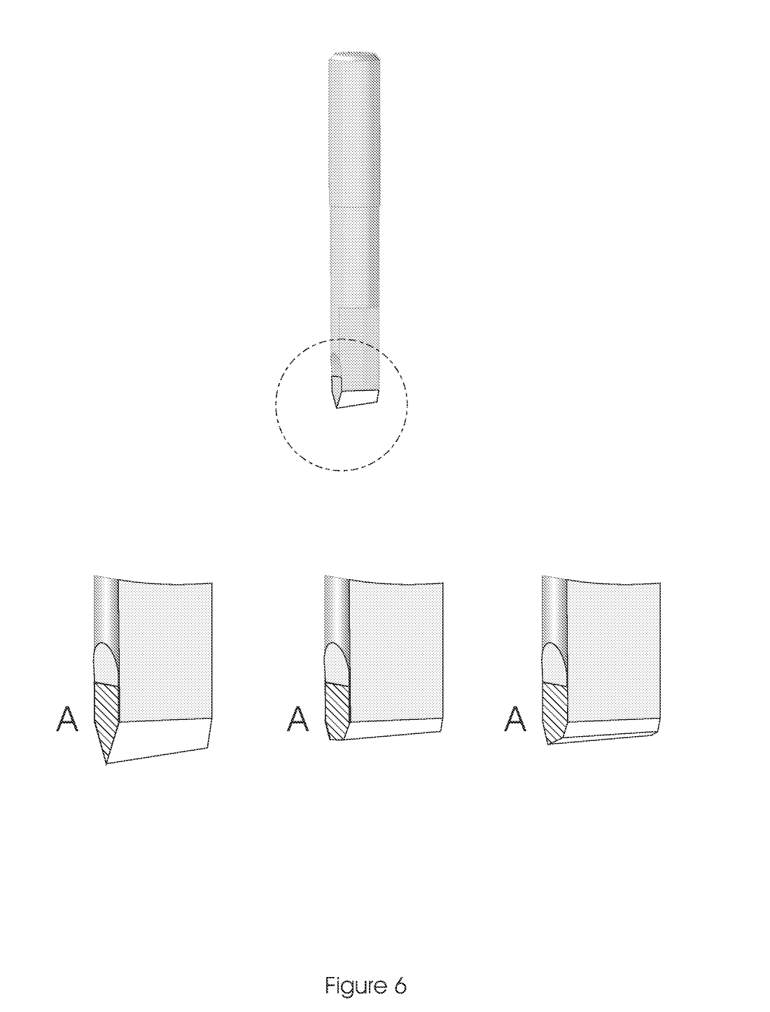

FIG. 6: shows several preferred embodiments of the cutting pins (4) with defined cutting surface and cutting edge. The cutting geometry is optimized for bone. Certain embodiments of these cutting pins have a sharp tip, like the one shown FIG. 6a, other embodiments have one straight cutting edges either exactly perpendicular to the axis of the cutting pin (FIG. 6b) or at an angle (FIG. 6c).

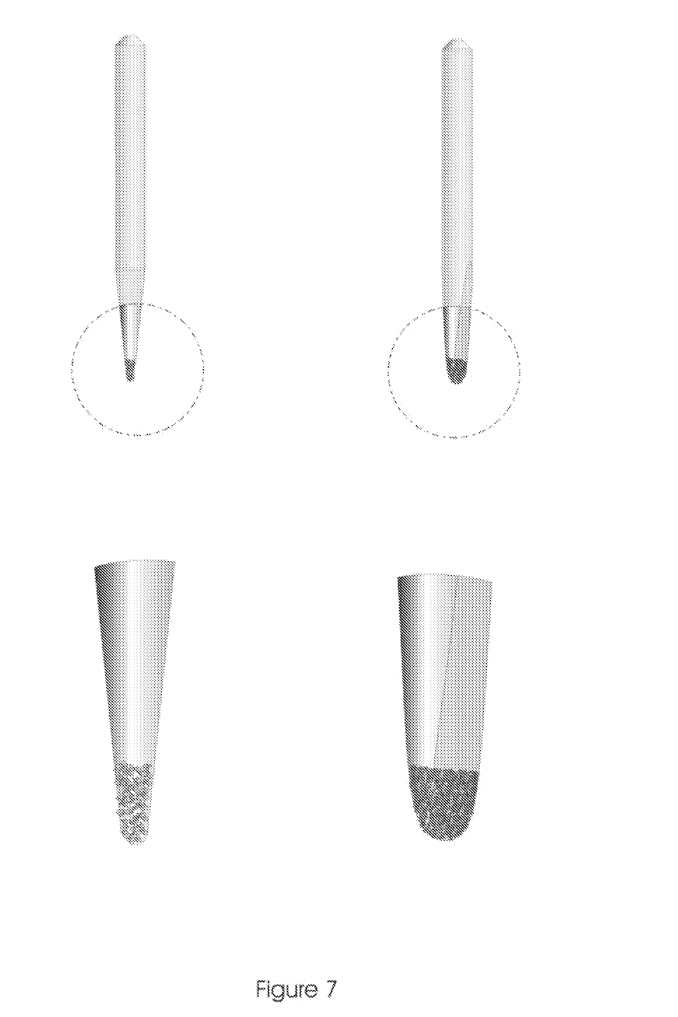

FIG. 7: shows several preferred embodiments of the cutting pins (4) with non-defined cutting surfaces- The version shown in FIG. 7a is coated with an abrasive medium on the front side. The one shown in FIG. 7b has a multitude of small jagged teeth producing the cutting channel in a manner similar to a rasp.

FIG. 8: shows the exact position and alignment of the cutting pin (4) on its corresponding smaller cogwheel (3).

The angular position of the hole for the cutting pin is positioned in a certain angel to the side of a tooth of the rolling cogwheel. This angel is the same for all rolling cogwheels. If the direction of view is exactly the direction of the axis, then the projection of surface A of the cutting pin is collinear with the radial beam (b). The distance between the center of the cutting edge and the center of the smaller cogwheel corresponds to “a” of the parameter Vₐ (see also point 2 of the description).

What we claim is:

1. A device to make openings in bone in the form of a bone lid comprising several cutting pins guided along the same hypotrochoid. The specific shape of the hypotrochoid is determined by the radius R of the fixed circle, the radius r of the rolling circle and the distance “a” of the curve generating point P from the center of the rolling circle (see FIG. 1).

2. Device as claimed in claim 1, in which the ratio V_{R} between the radius R of the fixed circle and the radius r of the rolling circle (V_{R}=R/r) is integer.

3. Device as claimed in claim 2, in which—depending on the actual embodiment—the ratio V_{R} has value of 3, 4, 5, 6, 7 or 8

4. Device as claimed in claim 1, in which—depending on the actual embodiment—the ratio Vₐ between the distance a of the curve generating point from the center of the rolling circle to the radius r of the rolling circle (Vₐ=a/r) has a value between 0,1 and 0,9.

5. Device as claimed in claim 1, in which—depending on the actual embodiment - the number of cutting pins has a value from 2 to 2. V_{R}-1.

6. Device as claimed in claim 1, in which the fixed circle is realized by a hollow cylinder with internal toothing. The pitch radius of this internal cogwheel is equal to R.

7. Device as claimed in claim 1, in which several cogwheel with external toothing roll inside this hollow cylinder with internal toothing. The pitch radius of each of these rolling cogwheels is r.

8. Device as claimed in claims 7, in which—depending on the actual embodiment—the number of rolling cogwheels has a value from 2 to 2. V_{R}-1.

9. Device as claimed in claim 6 and 7, in which the rolling cogwheels are attached to revolving support on which they can rotate freely and are forced to roll along the internal toothing of the hollow cylinder by the revolving movement of this support.

10. Device as claimed in claim 6, 7 and 8, in which the number of teeth of the internal toothing of the hollow cylinder is equal to or a multiple of the lowest common multiplier of V_{R}, the number of smaller cogwheels and the number of teeth of these smaller cogwheels.

11. Device as claimed in claim 7, in which each of the rolling cogwheels has attached exactly one cutting pin.

12. Device as claimed in claim 1, in which the shape and dimensions of the cutting pins are the same for all cutting pins.

13. Device as claimed in claim 12, in which each cutting pin has at its front side a defined cutting surface and cutting edge. The center of the cutting edge is the realization of the curve-generating point P.

14. Device as claimed in claim 12, in which each cutting pin has at its front side a multitude of non-defined cutting edges. The center of the tip of the pin is the realization of the curve-generating point P.

15. Device as claimed in claim 7, in which the distance between the curve generating point as defined in claims 13 and 14 and the center of the corresponding cogwheel is the same for all rolling cogwheels. This distance corresponds to “a” in the parameter Vₐ.

16. Device as claimed in claim 11, in which the angular position of the hole for cutting pin on the cogwheel it is attached to in relation to the toothing of this cogwheel is the same for of all smaller cogwheels.

17. Device as claimed in claim 13, in which the cutting surface of the cutting pin is oriented in such a way, that if the direction of view is exactly the direction of the axis, then the projection of the cutting surface is collinear with the radial beam from the center of the cogwheel to the center of the cutting edge.

18. Device as claimed in claim 9, in which the revolving support has on one end a drive shaft, driven by an external rotating drive mechanism and on the other end an axis or pin for centering the device.
